(19) United States
(12) Patent Application Publication
(10) Pub. No.: US 2023/0165905 A1
(43) Pub. Date: Jun. 1, 2023

(54) Stem Cells Delivery Device (SCDD) and method for Spinal Cord Injuries and diseases

(71) Applicant: Rassan Mohammad Tarabein, Daphne, AL (US)

(72) Inventor: Rassan Mohammad Tarabein, Daphne, AL (US)

(21) Appl. No.: 18/056,403

(22) Filed: Nov. 17, 2022

(51) Int. Cl. A61K 35/28; A61P 43/00; A61M 5/178; A61K 35/35

(52) U.S. Cl. CPC A61K 35/28; A61P 43/00; A61M 5/178; A61K 35/35; A61K 2035/124

(57) ABSTRACT

The Stem Cell Delivery Device comprises an Introducer, a Catheter, and Pressure Metered Injector Syringe. The device and method aim to deliver stem cells abundantly enough at the damaged site to allow recovery and restore function. The Method uses activated Stem Cells. It entails injecting via the Syringe Injector the epidural space with raw adipose tissue extract to circle the spinal cord about a level above and below the injured cord site till blockade of the subarachnoid space of the injured spinal cord site is achieved and radiographically verified. Then washed activated stem cells are injected via the Syringe into the blocked subarachnoid space to bridge between the upper and lower normal spinal tract, allowing the stem cells to grow new spinal tracts to connect and bridge over the damaged site. IV infusion of bone marrow stem cells is added to maximize the chance of recovery.

### BACKGROUND

About 250,000 people in the United States are paralyzed by Spinal Cord Injury (SCI) (population of 350 million), Half of this figure exists in Syria (20 million people) due to the ongoing 11 years long civil war. Despite the catastrophic impact, the current rate of spinal cord injury treatment failure is still high, probably due to the lack of a specific device or method to treat spinal cord injuries. The stem cells are currently infused intravenously or injected, often into the subarachnoid space. Hence the amount of stem cells that reach the damaged site is too little to restore a meaningful function.

### SUMMARY OF THE INVENTION

This invention and method allow stem cells used exclusively at the injury site to build new spinal tracts and bridge over the damaged site to connect and restore function. This is why many trials worldwide involved injecting Stem Cells into the spinal subarachnoid space or infused intravenously failed because too few stem cells reached the injured spinal site, never enough to promote a meaningful recovery.

### BRIEF DESCRIPTION OF THE DRAWINGS

FIG. 1: the “Introducer”, a curved tip with a stylet guide for easy steering, 2 mm diameter, comes in different lengths starting at 3.5 inches to 8 inches depending on the body habitus of the patient.

FIG. 2: the “Dacron Catheter”, a semi-stiff with a curved tip for easy steering and four apertures (holes) to allow delivery of thick materials (like adipose and stem cells). Caliber easily fits into the Introducer to slide through. Lengths 10-25 inches.

FIG. 3: the “Pressure Metered Syringe Injector” is a hard wall syringe to endure the pressure of injecting adipose and stem cells, also metered using the metric system (mm/cm).

FIG. 4: illustrates the catheter inserted into the introducer (after removing its stylet guide), showing how the catheter connects to the metered syringe injector.

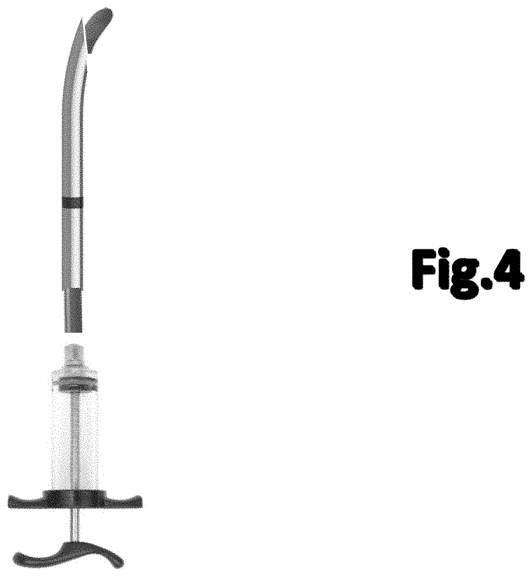

FIG. 5: illustrates the Method. A and C: adipose-washed cells are injected into the epidural space about a vertebral space above and below the damaged site; till a blockade of the subarachnoid space is achieved and verified radiographically. B: Washed and activated stem cells mixed with PRP (about 20%) are injected into the closed subarachnoid space above and below the damaged cord site connecting the normal spinal cord tissue before and after the damage site.

FIG. 6: depicting the treated spinal cord about six months after the procedure, showing how this device and method would give the stem cells being condensed and packed where they belong to grow new spinal tracts to reconnect the damaged spinal cord. The adipose tissue injected in the epidural space before and after the damaged cord site will be resorbed.

### DETAILED DESCRIPTION OF THE INVENTION

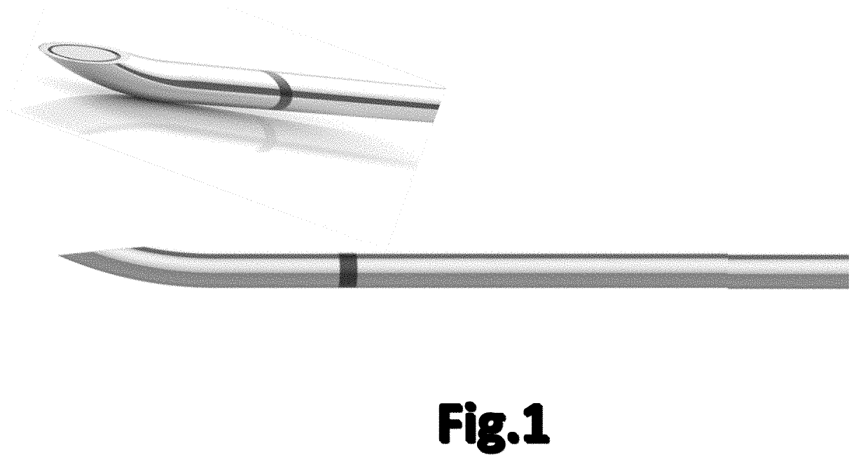

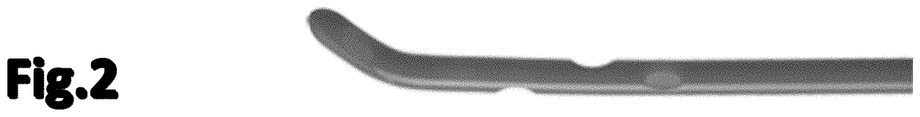

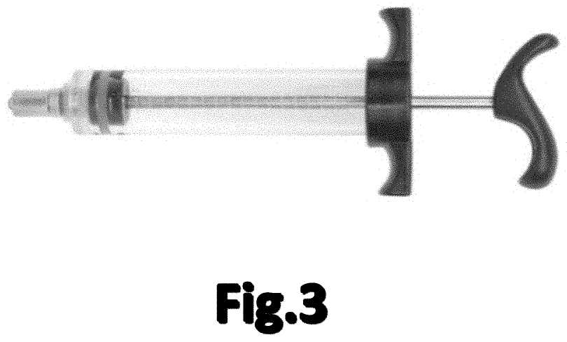

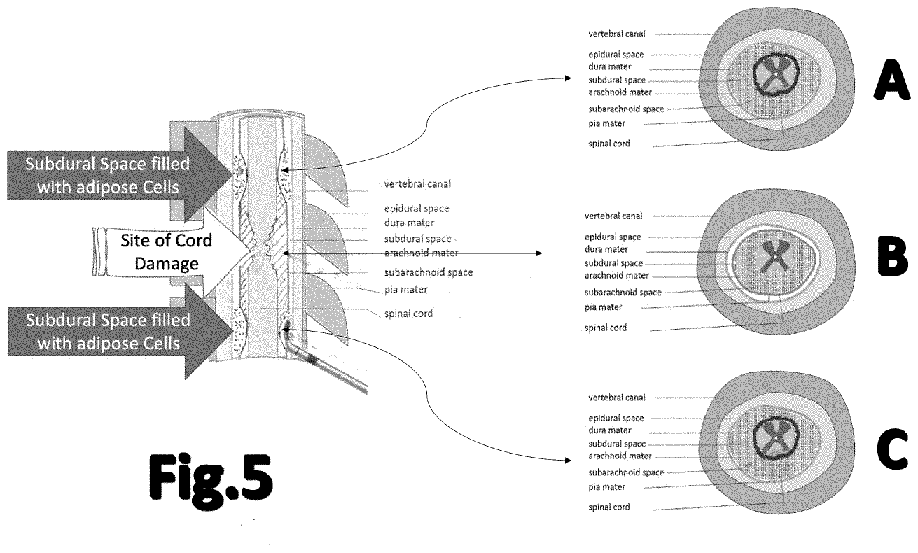

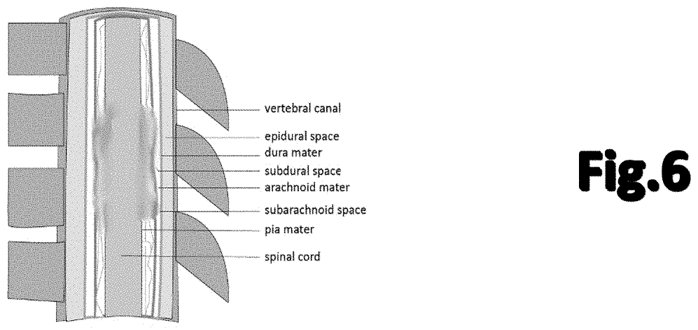

The method entails inserting the Introducer (FIG. 1) into the intervertebral space of the spine at about a level above and below the injury site. Then, the Stylet guide wire is pulled out, and the Dacron Catheter (FIG. 2) is inserted. The curved tip of the catheter enables the operator to steer it through the subdural space laterally till it hugs the cord and reaches the anterior aspects of the epidural space. Raw-washed packed adipose tissue derived from liposuction (after extracting stem cells) is injected via the Pressure Metered Syringe (FIG. 3) into the epidural space of the cord damage site to circle the spinal cord about a level above and a level below the injured spinal cord site till blockade of the subarachnoid space of the injured spinal cord site is achieved and radiographically verified (FIG. 5: A and C). Then washed activated stem cells mixed with 20% PRP are injected via the Pressure Syringe into the blocked subarachnoid space to bridge between the upper and lower normal spinal tract over the damaged site (FIG. 5: B), allowing the stem cells to grow new spinal tracts to connect and restore function, as depicted in (FIG. 6). The method includes IV infusion of bone marrow-derived stem cells as an adjunctive step to maximize the chance of recovery.

1. The Stem Cells Delivery Guide, as illustrated in FIG. 1.

2. The Stem Cells Dacron Catheter, as shown in FIG. 2.

3. The Method and procedure of Stem Cells Delivery for Spinal Cord Injuries and diseases as outlined in this application.
